(19) United States
(12) Patent Application Publication
(10) Pub. No.: US 2005/0031654 A1
(43) Pub. Date: Feb. 10, 2005

(54) Composition based on diosgenin ester for topical use

(76) Inventor: Michele Eymard, Meudon (FR)

Correspondence Address: BURNS DOANE SWECKER & MATHIS L L P, POST OFFICE BOX 1404, ALEXANDRIA, VA, 22313-1404, US

(21) Appl. No.: 10/497,030

(22) PCT Filed: Nov. 18, 2002
(86) PCT No.: PCT/FR02/03939

(30) Foreign Application Priority Data
Nov. 30, 2001 (FR) 01/15536

(51) Int. Cl. A61K 31/715; A61K 31/19

(52) U.S. Cl. 424/401; 514/557; 514/54; 514/574

(57) ABSTRACT

The invention concerns compositions useful in therapeutics and in cosmetology. The composition comprises a diosgenin ester with linear or branched alkylated ester chain containing 1 to 30 carbon atoms selected preferably among diosgenin lactate and glycalate. The invention is applicable in dermatology by acting on the superficial musculo-aponevrotic system or on estrogen and progestenone cell receptors.

The present invention relates to a composition which is useful in human or animal therapeutics and cosmetology, and more particularly to a composition with local action which can be applied topically and which comprises a diosgenin ester.

Diosgenin, a structural analog of cholesterol, is essentially used in therapeutics because of its anti-inflammatory properties and its action in compensating for the deleterious effects of estrogens, more particularly in the perimenopausal and menopausal period. It is also used in cosmetics and in dermatology for its antiseptic properties on the skin.

Diosgenin can be extracted from plants of the Dioscorea genus, in particular Dioscorea opposita, which is a plant used in traditional Chinese medicine. The extraction can be carried out by the usual techniques, for example by ethanolic extraction, from any part of the plant, and preferably the rhizome, which has been pre-dried and ground. A method for growing plants of the Dioscorea family, in particular Dioscorea floriburida and Dioscorea composita, which makes it possible to improve the diosgenin content is described in patent U.S. Pat. No. 3,918,200.

Patent FR 2,367 M describes the use of diosgenin as a medicinal product exhibiting antiphlogistic and anti-inflammatory activity which is useful for the treatment of rheumatic and vascular conditions. Patent FR-A-2,786,097 describes cosmetic slimming compositions comprising approximately 5% of extracts of Dioscorea opposita, having the effect of acting on the lipid metabolism of adipocytes by limiting fat accumulation in the cell. Patent U.S. Pat. No. 5,804,211 describes a plant-based composition in the form of a nasal lotion, intended to limit or eliminate snoring, and containing an extract of Dioscorea, ginger, chamomile and a potassium or sodium salt. The extract of Dioscorea is preferably Dioscorea villosa, used in a proportion of 0.01 to 0.4% by weight relative to the total weight of the composition.

The use of diosgenin in therapeutic compositions has also been described, for example, in patent EP-A-873,125 relating to a composition combining diosgenin with vitamins, such as vitamin K and vitamin D, which can be administered orally or transdermally, for treating osteoporosis.

It is also known that diosgenin, a steroidal saponin, is a precursor of various steroids, and in particular of DHEA, which is an important hormone in the human or animal body, produced by the adrenal glands, and which is converted to estrogen and testosterone. However, diosgenin is not converted directly to progesterone, but to a hormone regulator. Nothing in the scientific literature makes it possible to suppose that diosgenin, in particular when it is administered topically, could have the same activity as DHEA, in particular because the enzymes capable of converting it in the body have never been demonstrated. On the other hand, it is possible to prepare DHEA from diosgenin in the laboratory.

Clinical studies carried out by the applicant have made it possible to show that diosgenin derivatives, in particular diosgenin esters, have properties which are useful in dermatology and in cosmetics, and in particular an action on the superficial musculoaponeurotic system when they are given by external or transdermal topical administration.

The subject of the present invention is therefore a composition which can be used topically in therapeutics and in cosmetology, comprising a diosgenin ester.

A subject of the invention is also a cosmetic and/or dermatological composition with a given content of diosgenin ester.

Finally, a subject of the invention is the use of a diosgenin ester for producing a dermatological medicinal product which acts, by means of an action similar to that of progesterone and of estrogens, on the superficial musculoaponeurotic system or on cellular estrogen and progesterone receptors, for the treatment of skin conditions, and also a cosmetic treatment process for the skin.

A subject of the present invention is also a composition having an intracellular or extracellular adaptogenic effect on the receptors of cells of the epidermis, of the dermis of the superficial musculoaponeurotic system, and of the hypodermis, and allowing application thereof in dermatology and in cosmetics, in particular for regenerating and restructuring the skin and, in general, for combating the signs of skin aging.

The cosmetic and/or dermatological composition of the invention is distinguished in that it contains a diosgenin ester comprising a linear or branched alkylated ester chain of 1 to 30 carbon atoms.

The ester residue carried by the diosgenin can come from an acid preferably containing 1 to 30 carbon atoms, and for example a fatty acid, an amino acid or alternatively an alpha-hydroxy acid. The fatty acid may be, for example, stearic acid, oleic acid or linoleic acid; the amino acid is preferably chosen from leucine, proline, thymidine, glucosamine and serine; the alpha-hydroxy acid is preferably chosen from glycolic acid, lactic acid, mandelic acid and citric acid. It may be most particularly advantageous to use diosgenin lactate or glycolate.

According to a variant in accordance with the present invention, an alpha-hydroxy acid such as glycolic acid, lactic acid, thiolactic acid or thioglycolic acid, or one of their salts, is used together with the diosgenin ester.

In accordance with the present invention, it is particularly advantageous to use the diosgenin ester in combination with a mucopolysaccharide, in particular a hydrolyzed mucopolysaccharide.

The studies carried out have shown that the diosgenin esters according to the invention, applied topically, for example in the form of a gel, cream or lotion, penetrate more effectively into the epidermis and exhibit an improved bioavailability compared with pure diosgenin.

The diosgenin ester can be prepared according to the usual techniques by reacting diosgenin with the corresponding acid, under suitable operating conditions. The diosgenin can itself be obtained from an extract of plants of the family Dioscoreaceae.

The extract of Dioscorea used in the present invention for preparing a diosgenin ester can be any extract of Dioscorea of the family Dioscoreaceae and, for example, Dioscorea opposita, Dioscorea composita, Dioscorea floriburida, Dioscorea villosa, Dioscorea hypoglauca, yam or wild yam, and preferably Dioscorea opposita. It is obtained in the form of an extract of the entire plant or, preferably, of the rhizome, as a dry extract or essential oil. As indicated above, the diosgenin ester can be prepared by any suitable process, by means of the action of a suitable acid on the diosgenin or on the extract of Dioscorea having a satisfactory diosgenin content.

As indicated above, the compositions according to the present invention contain a given content of diosgenin ester, which can be relatively high compared to the usual doses of dermatological active principles, and can be between 0.05% and 80% by weight relative to the total weight of the composition, and preferably between 0.5% and 30% by weight. These ester contents can be chosen as a function of the method of administration envisioned. For example, in the case of a patch for transdermal administration, the content of diosgenin ester can be advantageously between 15 and 30% by weight.

The adaptogenic effect of the composition of the invention results from the high content of diosgenin ester providing an effective action which results in an activation of the effect similar to that of the progesterone in the body, and a modulation of the cellular estrogen and progesterone receptors.

In particular, the composition of the invention exerts its effects on the superficial musculoaponeurotic system and on all the tissues in general, by causing an increase in the sensitivity of the membrane or cytoplasmic receptors of the cells to circulating hormones. This effect therefore makes it possible to compensate for the decrease in the number of receptors by an increase in their sensitivity. The superficial musculoaponeurotic system is a connective fibrous network containing fibroblasts sensitive to circulating estrogens and muscle fibers sensitive to progesterone.

This action is particularly effective on skin exhibiting signs of skin aging, by compensating for the hormonal loss by means of an increase in activity associated with the increase in receptor sensitivity.

This activity can be advantageously exploited in treating the signs of skin aging, by causing an increase in the thickness of the skin by means of densification of the structure of the epidermis, of the dermis and of the hypodermis, and also of the dermis of the superficial musculoaponeurotic system. The signs of skin aging are more particularly those which are characterized by a relaxation of the structure of the skin and a decrease in the thickness of the skin and, as indicated above, the composition according to the invention acts by compensating for the hormonal loss by increasing the sensitivity of the estrogen and progesterone receptors. This activity can also be taken advantage of in dermatological and cosmetic surgery treatments, by providing densification and a restructuring of the skin and an increase in muscle tension, in particular in the face.

The composition of the invention can also be used advantageously for treating various skin conditions and, for example, for treating burns and cicatrization, and for an anti-edematous action in a combined treatment for, for example, venous insufficiency or localized adiposis.

The use of thiolactic acid or of thioglycolic acid, or one of their salts, in combination with the diosgenin ester according to the invention proves to be particularly effective when used for straightening keratin fibers, eyelashes or the hair.

Finally, the composition according to the invention is useful in the treatment of excess weight. In fact, estrogens and progesterones are the known hormonal agents of obesity of the limbs of the human body. In the adipocytes, the distribution of α-adrenergic receptors and of β-adrenergic receptors is approximately 85/15. Furthermore, it is known that the antero-internal face of the thigh comprises type I fats having particular sensitivity to estrogens and a high lipoprotein lipase activity, while the external face of the thigh has a particular sensitivity to progesterone, the anti-edematous action of which is known. Thus, problems of hormonal regulation can cause a decrease, or even a disappearance, of the β-adrenergic receptors which promote lipolysis more particularly in the pelvic region and on the inside of the knees, which can result in deformities in these regions. In addition, vascular disorders can further promote the disappearance of the β-receptors. As a result of this, there is an accumulation of fat on the internal face of the thighs, in particular in a hyperestrogenic woman exhibiting circulatory problems.

The composition according to the invention makes it possible to combat this weight excess by reestablishing cutaneous trophism and a normal microcirculation allowing the release of certain β-adrenergic receptors. The reestablishment of lipolysis and of the perinodal microcirculation is demonstrated by an increase in the amplitude and a modification of the plethysmographic plots.

The diosgenin esters according to the present invention can be formulated according to the usual techniques and can be administered in the form of emulsions, gels, masks, lotions, solutions for spraying, etc. The emulsions can be of the oil-in-water (O/W) or water-in-oil (W/O) type, for example gels or creams. Use may also be made of encapsulation techniques and, for example, of the technique of encapsulation in liposomes.

The emulsifier used for the composition of the invention is chosen as a function of the formulations and of the conditions for administration. For example, the emulsifier can be chosen from high molecular weight carboxyvinyl polymers (for example Carbopol®), polysorbates (for example Tween 200 or Tween 600), sorbitan esters and, in particular, a sorbitan monostearate, tristearate, monopalmitate and laurate (for example the emulsifiers known under the trademark Arlacel®). Use may also be made of other emulsifiers, such as various derivatives of stearic acid or palmitic acid, and, for example, PEG 1000 stearate or PEG 20® stearate or a C₁₂-C₂₀ ester of PEG 8 or PEG 9, stearic or palmitic acid glycerides, glycerylstearate, a polyethylene glycol stearate, a steareth or a ceteareth (for example ceteareth 20), or else polyglyceryl-2 sesquioleate, polyoxyethylene cetyl ether, or an emulsifiable silicone.

Besides the emulsifiers mentioned above, the compositions can contain various usual adjuvants chosen from those used in techniques for preparing cosmetic and dermatological compositions, and for example preserving agents, thickeners, hydrophilic or lipophilic gelling agents, antioxidants, moisturizers, surfactants, fragrances, dyes, pigments, fillers and various additives intended to improve the physical properties of the composition. It may also be advantageous to incorporate into the composition of the invention sun-screening or sun-filtering agents which are active in the UVA and/or UVB range, chosen as a function of the desired degree of protection.

The diosgenin esters according to the present invention can be used for producing a dermatological medicinal product acting by means of an action on the superficial musculoaponeurotic system at the level of the cellular receptors, for the treatment of various skin disorders, and also in a cosmetic treatment process for the skin, consisting in applying to the area to be treated an effective amount of a diosgenin ester, alone or in combination with acceptable excipients and supports.

The following examples illustrate the invention in greater detail without limiting the scope thereof.

### EXAMPLE 1

Using the usual techniques of dermatological formulations, an exfoliating gel having the following composition, indicated in part by weight, is prepared:

| | |
|---|---|
| diosgenin lactate | 1.0 |
| glycolic acid | 70.0 |
| moisturizer | 5.0 |
| lysine pyrrolidonecarboxylate | 4.0 |
| ethanol | 5.0 |
| ammonium hydroxide | 5.0 |
| preserving agent | 3.0 |
| fragrance | 0.5 |
| water | qs 100.0 |

This exfoliating gel can be used by application to the skin once a week. The trials carried out demonstrated an evident antiwrinkle and restructuring effect from the end of the second week of application to areas exhibiting the signs of skin aging consisting of wrinkles, a thinning of the skin and a relaxing of the skin. A tensioning effect on the structure of the skin is notably observed from the first week of treatment by means of the action on the superficial musculoaponeurotic system.

### EXAMPLE 2

A slimming and anti-edematous cream having the composition by weight indicated below is prepared.

| | |
|---|---|
| diosgenin glycolate | 5.0 |
| hydrolyzed mucopolysaccharides | 2.0 |
| ethanol | 5.0 |
| moisturizer | 5.0 |
| C₁₂₋₂₀ PEG-8 ester | 7.0 |
| cetearyl/palmitoyl octanoate | 7.0 |
| silk amino acids | 2.0 |
| extract of ginko biloba | 1.0 |
| troxerutin | 1.0 |
| preserving agent | 0.2 |
| fragrance | 0.1 |
| water | qs 100.0 |

This cream was applied once or twice a day to the legs of 30- to 50-year-old patients, on the thighs and the knees.

The anti-edematous action is visible from the second day of treatment. Tissue drainage is particularly noticeable after liposuction of the thighs and the knees, preventing retractile fibrosis. In addition, the action on the superficial musculoaponeurotic system is notable by virtue of the very rapid tensioning of the tissue, which makes it possible to apply this treatment to localized adiposis. Prolonged action results in a decrease in the excess weight due to the adaptogenic effect which reactivates the progesterone-sensitive receptors.

### EXAMPLE 3

A composition according to the present invention, which promotes hair regrowth, having following composition by weight, is prepared.

| | |
|---|---|
| diosgenin glycolate | 20.0 |
| hydrolyzed mucopolysaccharides | 0.5 |
| C₁₂₋₂₀ PEG-8 ester | 8.0 |
| cetearyl octanoate | 2.0 |
| propylene glycol dicaprylate dicaprate | 3.0 |
| aescin | 0.5 |
| extract of ginko biloba | 1.0 |
| preserving agent | 0.2 |
| water | qs 100.0 |

1. A composition which is useful in therapeutics and in cosmetology, for topical application, which comprises a diosgenin ester comprising a linear or branched alkylated ester chain of 1 to 30 carbon atoms.

2. The composition as claimed in claim 1, wherein the diosgenin ester comprises diosgenin lactate or diosgenin glycolate.

3. The composition as claimed in claim 1, wherein the content of diosgenin ester is between 0.05% and 80% by weight relative to the total weight of the composition.

4. The composition as claimed in claim 1, which also comprises an alpha-hydroxy acid or a salt of an alpha-hydroxy acid.

5. The composition as claimed in claim 4, wherein the alpha-hydroxy acid is selected from the group consisting of glycolic acid, lactic acid, mandelic acid, citric acid, thiolactic acid and thioglycolic acid.

6. The composition as claimed in claim 1, which also comprises a mucopolysaccharide.

7. (Canceled)

8. A cosmetic treatment process for the skin, consisting in applying to the area to be treated an effective amount of diosgenin ester as claimed in claim 1.

9. The composition as claimed in claim 2, wherein the content of diosgenin ester is between 0.05% and 80% by weight relative to the total weight of the composition.

10. A method of treating skin conditions by applying the composition of claim 1 to thereby exert an action on the superficial musculoaponeurotic system or on the cellular estrogen and progesterone receptors.
